(19) United States
(12) Patent Application Publication
(10) Pub. No.: US 2022/0330879 A1
(43) Pub. Date: Oct. 20, 2022

(54) SYSTEMS AND METHODS FOR MONITORING THE STATE OF A DISEASE USING A BIOMARKER, SYSTEMS AND METHODS FOR IDENTIFYING A BIOMARKER OF INTEREST FOR A DISEASE

(71) Applicants: Aldo FAISAL, London (GB); Richard FESTENSTEIN, London (GB); Valeria RICOTTI, London (GB); Thomas VOIT, London (GB)

(72) Inventors: Aldo Faisal, London (GB); Richard Festenstein, London (GB); Valeria Ricotti, London (GB); Thomas Voit, London (GB)

(21) Appl. No.: 17/764,887

(22) PCT Filed: Oct. 2, 2020
(86) PCT No.: PCT/EP2020/077703

§ 371 Date: Mar. 29, 2022

(30) Foreign Application Priority Data
Oct. 2, 2019 (GB) 1914193.6

(51) Int. Cl. A61B 5/00; C12Q 1/6883; A61B 5/11; G16H 50/30; G16H 40/63

(52) U.S. Cl. CPC A61B 5/4082; C12Q 1/6883; A61B 5/112; G16H 50/30; G16H 40/63; C12Q 2600/112; C12Q 2600/158; C12Q 1/6851

(57) ABSTRACT

A system for monitoring the state of a disease or the state of a person using a biomarker, the system comprising: a motion data obtaining unit configured to obtain from at least one motion sensor motion data from a person having the disease, a generating unit configured to generate a value of the biomarker of the disease based on the obtained motion data, and an assessing unit configured to assess the value of the biomarker of the disease and, based on the assessment, to output information related to the state of the disease or state of a person

### TECHNICAL FIELD OF THE INVENTION

The present invention relates to biomarkers and to sensor-diagnostic and monitoring technology for diseases including neuromuscular/degenerative, neurological, neurodegenerative or polygenic diseases or disorder, and in general genetic or acquired diseases or disorders, and more in general to biomarkers for detecting a state of a person (e.g. for measuring human performance).

### BACKGROUND OF THE INVENTION

The main way our brain and body interact with the physical world is through the generation of movements using various body parts. Ageing and various degenerative disorders (such as Parkinson's disease (PD), Alzheimer's disease (AD), Motor Neuron Disease (MND), Huntington's disease (HD) etc.) adversely affect the way human brain and the body can operate, thus most disorders subtly alter the control of movement (e.g. ataxia), its execution (e.g. muscular dystrophy) or the mental structuring movement (e.g. dementia). Background of the invention is provided that was evaluated in biomedical engineering work for two different types of diseases, Friedreich's ataxia and Duchenne Muscular Dystrophy.

### Neurological Disease

Friedreich's ataxia (FRDA) is the commonest inherited ataxia. Friedreich's ataxia is an inherited neurodegenerative disease with slow progression that affects the central nervous system causing initially subtle changes in patient's behaviour that worsen over time. It typically starts manifesting itself during childhood and the main symptoms include poor balance (discordination), gradual loss of strength and sensation in the limbs, muscle stiffness (spasticity), curvature of the spine (kyphoscoliosis), impaired speech, hypertrophic cardiomyopathy and it has also been associated with diabetes, bladder control difficulty (incontinence), impaired vision and hearing loss. In addition, they frequently develop heart problems. These symptoms often have a serious effect on their ability to perform everyday tasks. The difficulty is that because the disease only gets worse very slowly it is necessary to perform this study on a large number of patients over a long period of time.

The currently used clinical scales that quantify the progression of neurodegenerative diseases such as Friedreich's ataxia (FRDA) have been shown to lack objectivity and are insensitive to the extremely slow progression of such diseases. This means that it might take months before they reveal any change. Consequently, there is a clear need for the development of more accurate and sensitive digital biomarkers that can detect even small changes in patient performance. Currently, the only way of evaluating the progression of FRDA disease in patients is through a series of score-based behavioural tests evaluated by-eye. The most commonly used tests are the Scale for the Assessment and Rating of Ataxia (SARA), the Inventory of Non-Ataxia Signs (INAS), Spinocerebellar Ataxia Functional Index (SCAFI) and Friedreich's Ataxia Rating Scale (FARS). All these scales can assess patient's motor control and coordination skills through a series of evaluation tests, such as the 8 Meter Walk (8MW), the 9-Hole Peg Test (SHPT), the finger-nose test, the finger tapping test, the heel-shin-slide test. Though these score-based metrics are able to quantify the ataxia disease, they are still subject to some variability due to the subjective estimates of some of their components. This variability can be reduced with extensive training of the clinicians. However, this still does not eliminate the effect of the patient's physical and psychological state on the output of the tests. These metrics also suffer from low sensitivity resulting in longer periods (2 years with 184 patients or 1 year with 548 patients) to measure changes in the behaviour in FRDA creating the need for much larger study groups in clinical. The lack of precision biomarkers of progression is due to the complex nature of FRDA related behavioural change.

### Neuromuscular Disease

Duchenne muscular dystrophy (DMD) is a known progressive genetic disorder that is characterised by muscle degeneration. The defected gene which leads to DMD is associated with the production of a protein called dystrophin which supports muscle fibre strength, the absence of dystrophin is the root cause of muscle weakness in DMD patients. DMD mainly affects young boys with symptom onset around the age of five leading to limited quality of life and a greatly shortened life expectancy. There is currently no cure for DMD and there are a limited number of approved therapies that aim to treat the underlying cause. For this reason, a large number of experimental treatments and clinical trials are currently being tested. Scientists from around the world are researching methods for reducing the detrimental effects of DMD, ranging from developing a synthetic alternative to dystrophin to protecting muscles from injury.

Clinical trials for Duchenne muscular dystrophy (DMD) assess functional performance in artificial set-ups, relying on subjective and motivation-dependent tasks. Furthermore, current outcome measures are subject to intra-rater & inter-rater variability in the patient performance itself, making drug development challenging. As a consequence, a larger sample size and longer duration of clinical trials are required, often delaying access to novel therapies. Because clinical endpoints in DMD rely on motivation-dependant assessments performed in clinic, they may not adequately reflect disease state and therapy impact on every-day life functionality, which in turn is a more accurate reflection of impact on quality of life. The assessment of disease progression and potential response to treatment are generally considered very challenging.

Current methods used to measure the severity of medical conditions that affect the body's motor responses or muscle strength are an area of constant discussion. They are expected to be non-invasive, easy to carry out and cheap whilst providing a clinically meaningful endpoint. The two most commonly used tests which track the progression of DMD include the Six Minute Walk Test (6MWT) and the North Star Ambulatory Assessment (NSAA) Scale. Both of these assessments are clinically meaningful, however, they also have limitations. They are subjective to the marker, dependent on the patient's mood and may not provide precise or perceptive information. Further to this, it is likely that these tests capture redundant information whilst being a distressing experience for the patient.

### SUMMARY OF THE INVENTION

In view of the above discussion, there is a need to establish a relevant and objective biomarker or clinical endpoint to act as an indication of a patient's response to a trial (e.g.: a therapy or exercise scheme), in particular in the field of neuromuscular/degenerative, neurological, neurodegenerative or polygenic or skeletal disease (e.g. osteoarthritis) or cardiovascular disease or cancer (brain or bone tumors), respiratory diseases (e.g. COPD)) or other genetic or acquired diseases.

Thus, an object of the present invention is to provide an accurate and reproducible biomarker which can track the progression of a condition and may lead to faster and more objective development and/or validation of potential therapies. More generally, the present invention aims at providing a biomarker which can be used to properly detect the state of a person, such as e.g. the state of a disease such as a neuromuscular condition.

A further goal of the present invention work is to provide a procedure to derive new digital biomarkers, which are suitable to monitor the state of a person, e.g. the state of disease. A further goal of the invention is to provide a procedure to derive new digital biomarkers, which are independent from artificial set-up and subjective biases, to capture individual variations in disease progression and benefit to therapies.

In view of the above objects, the present invention proposes a method for identifying a biomarker of interest for a disease, the method comprising:

(1) determining a candidate biomarker,

(2) obtaining from at least one motion sensor first motion data from at least one person having the disease,

(3) obtaining from the at least one motion sensor second motion data from at least one person not having the disease,

(4) calculating at least a first value related to the candidate biomarker based on the first motion data and at least a second value related to the candidate biomarker based on the second motion data;

(5) in a first determination step, determining based on the first and the second values of the candidate biomarker whether the candidate biomarker distinguishes between the at least one person having the disease and the at least one person not having the disease,

(6) obtaining first clinical score data according to a conventional clinical score protocol from at least one person having the disease,

(5) obtaining second clinical score data according to the conventional clinical score protocol from at least one person not having the disease,

(7) determining a level of correlation between a set of the calculated first and second values of the candidate biomarker and a set of the obtained first and second clinical score data,

(8) identifying, based on the result of the first determination step and on the level of correlation, whether the candidate biomarker is a biomarker of interest.

Furthermore, the present invention provides a system for monitoring the state of a disease using a biomarker, the system comprising:

a motion data obtaining unit configured to obtain from at least one motion sensor motion data from a person having the disease,

a generating unit configured to generate a value of the biomarker of the disease based on the obtained motion data, and

an assessing unit configured to assess the value of the biomarker of the disease and, based on the assessment, to output information related to the state of the disease.

Therefore, the novel methodology according to the invention enables the precise capture of human activity and the observation of deviations from a baseline in a quantitative or qualitative manner. The degree of variation from the baseline embodies a biomarker for the objective assessment of diseases or human state (biomarkers) or for providing direct feedback on rehabilitation or wellbeing or training methods

Based on results initially gained in the context of monitoring Friedreich's ataxia (FRDA) and Duchenne Muscular Dystrophy (DMD), according to the invention, on a more general level, methods are developed for measuring how severe a condition is in individual patients as a disease progresses, to reduce the number of patients needed in a trial to meet such a biomarker endpoint and make more relevant and unbiased assessments than current methods.

### DETAILED DESCRIPTION OF THE PREFERRED EMBODIMENTS

Other features and advantages of the present invention will become more apparent from the description of a preferred but not exclusive embodiments of a method for identifying a biomarker of interest for a disease, and of a system for monitoring the state of a disease using a biomarker according to the present invention, illustrated by way of a representative, non-limiting example in the appended drawings, in which:

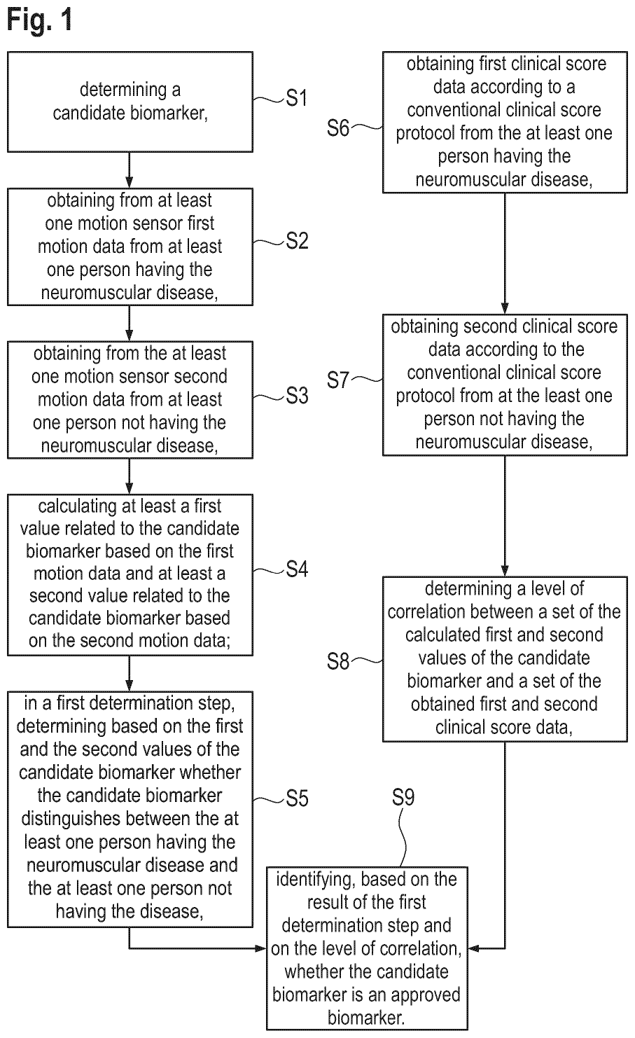

FIG. 1 is a schematic representation of a method to identify a biomarker of interest according to the invention;

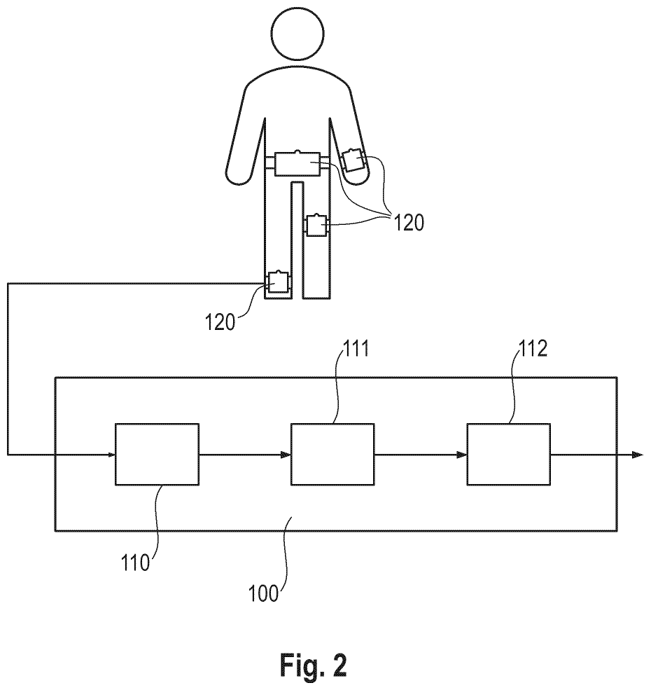

FIG. 2 is a schematic representation of a system for determining the state of a disease according to the invention;

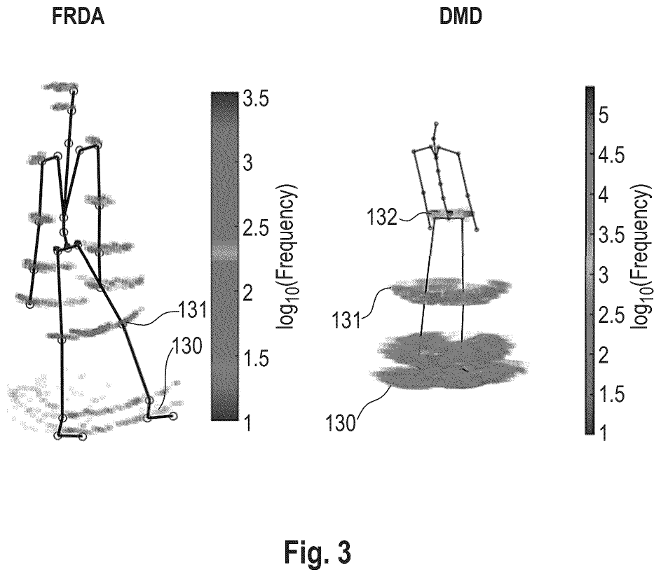

FIG. 3 shows biomarkers such as workspace volume for FRDA and DMD;

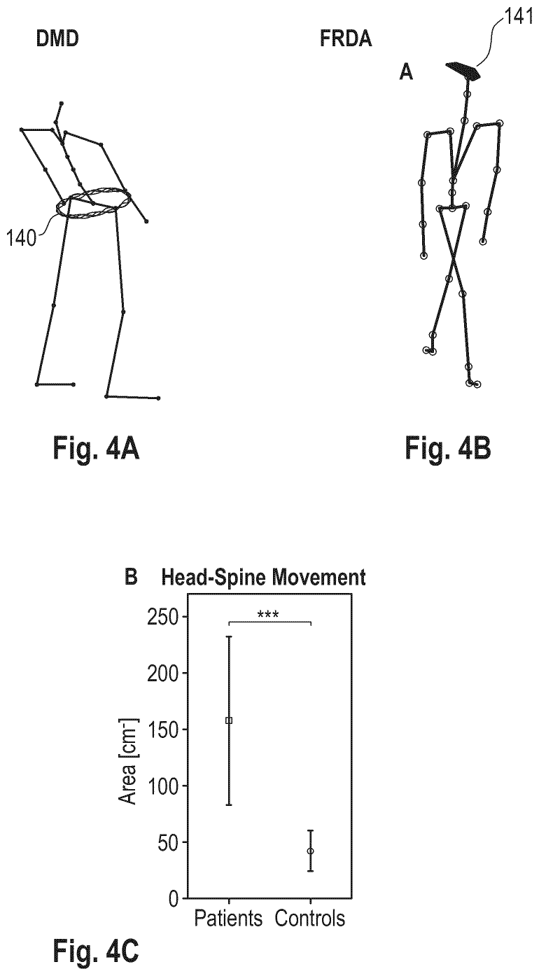

FIGS. 4A and 4B show biomarkers, such as head orbit (FRDA) and hips orbit area (DMD) and FIG. 4C shows the difference between patient and controls in FRDA;

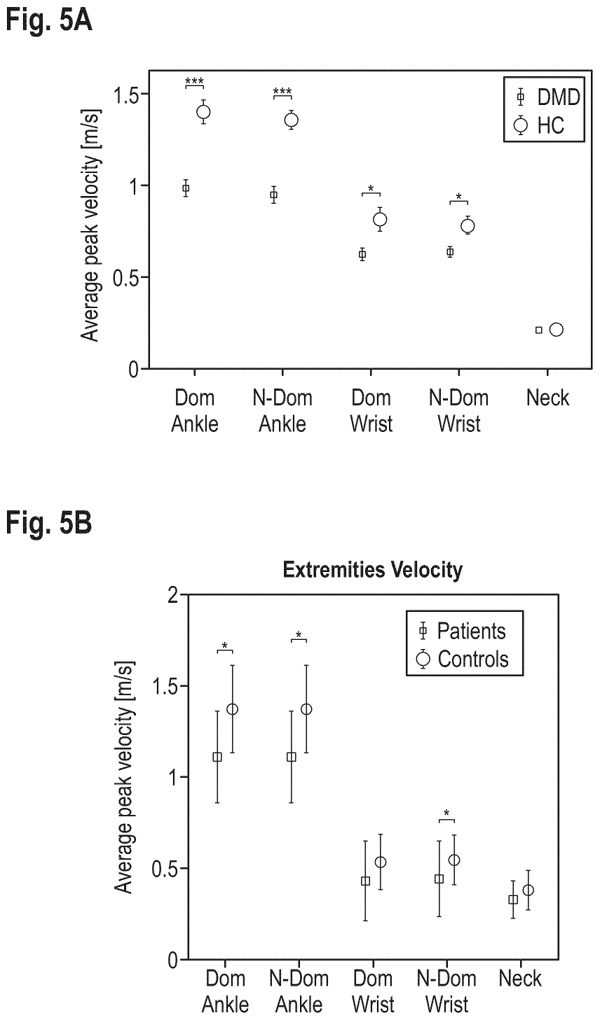

FIG. 5A shows a graph of the average peak velocity for various sensor locations for an experimental DMD group and a control group;

FIG. 5B shows a graph of the average peak velocity for various sensor locations between patient and controls in FRDA;

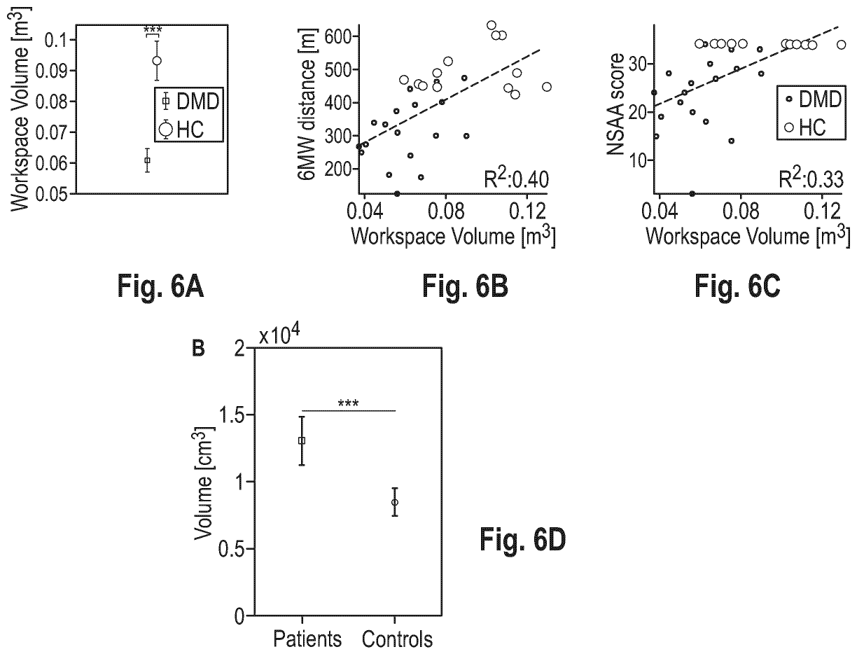

FIG. 6A shows the workspace volume during walking for an experimental DMD group and a control group;

FIG. 6B shows measurements of 6MW distance and workspace volume for an experimental DMD group and a control group, including a linear regression;

FIG. 6C shows measurements of NSAA total score and workspace volume for an experimental DMD group and a control, including a linear regression;

FIG. 6D shows the workspace volume during walking for an experimental FRDA group and a control group;

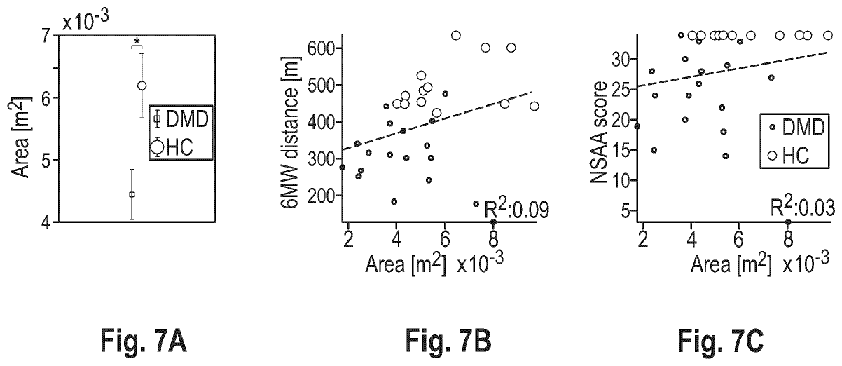

FIG. 7A shows the area of a hip orbit for an experimental DMD group and a control group;

FIG. 7B shows of 6MW distance and area of a hip orbit for an experimental DMD group and a control group, including a linear regression;

FIG. 7C shows measurements of NSAA total score and area of a hip orbit for an experimental DMD group and a control, including a linear regression;

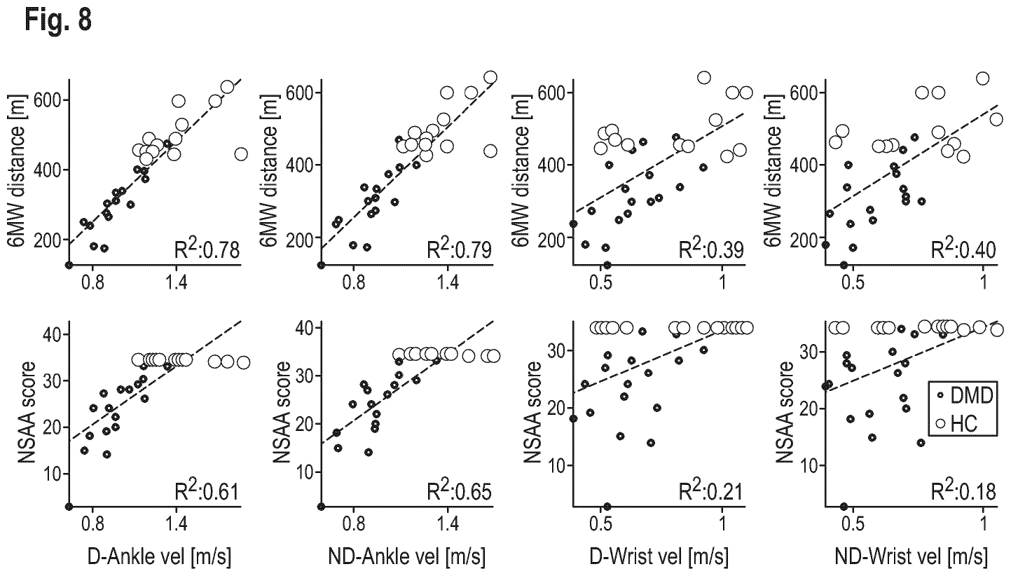

FIG. 8 shows various graphs of measurements points of a clinical score and a measured velocity for an experimental DMD group and a control group;

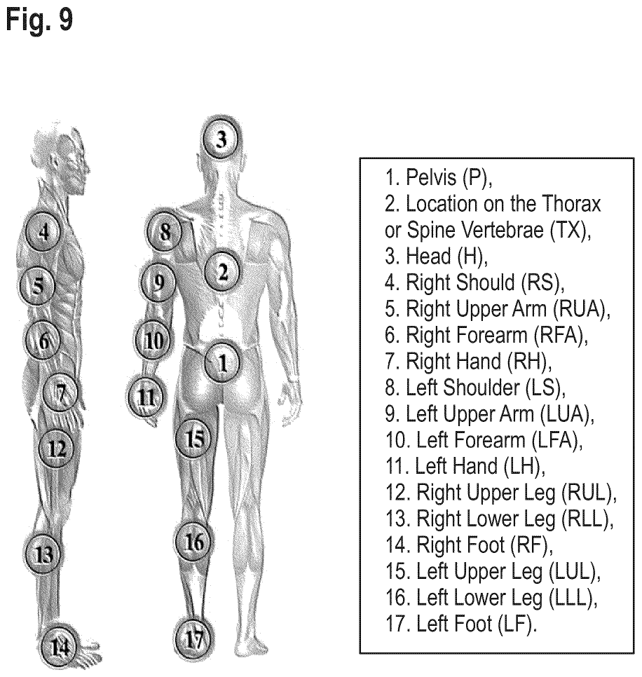

FIG. 9 shows a side view (left) and a back view (right) of a person, indicating various sensor placement positions;

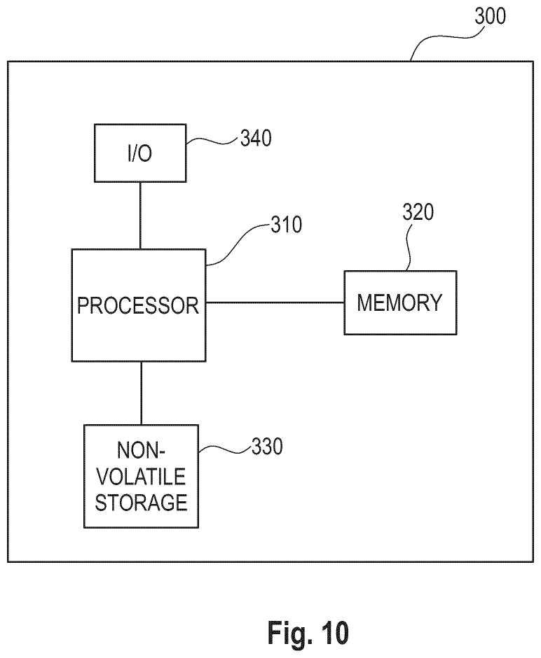

FIG. 10 shows an illustrative implementation of a computer system which can be used to implement the system and carry out the method according to the present invention.

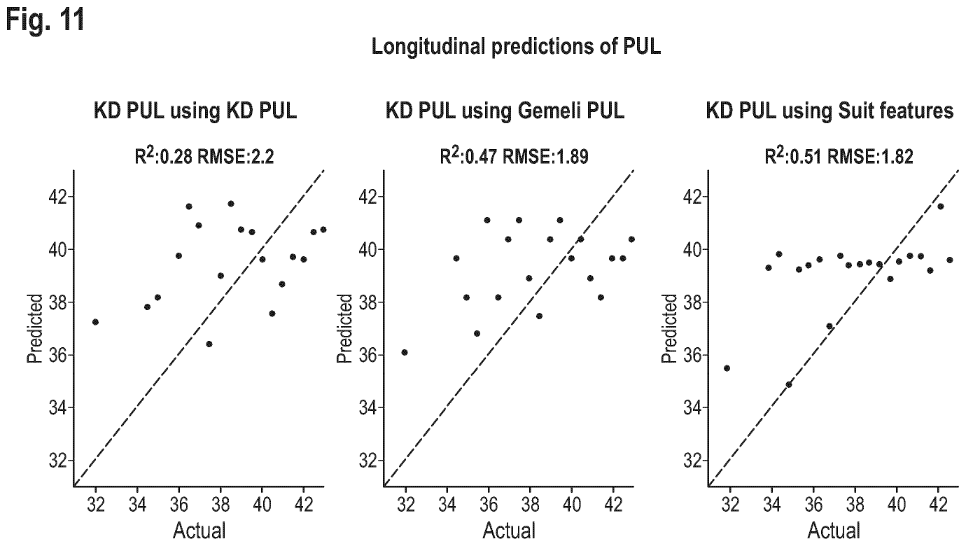

FIG. 11 shows the longitudinal prediction of PUL, using respectively KD PUL, Gemeli PUL and the suit features.

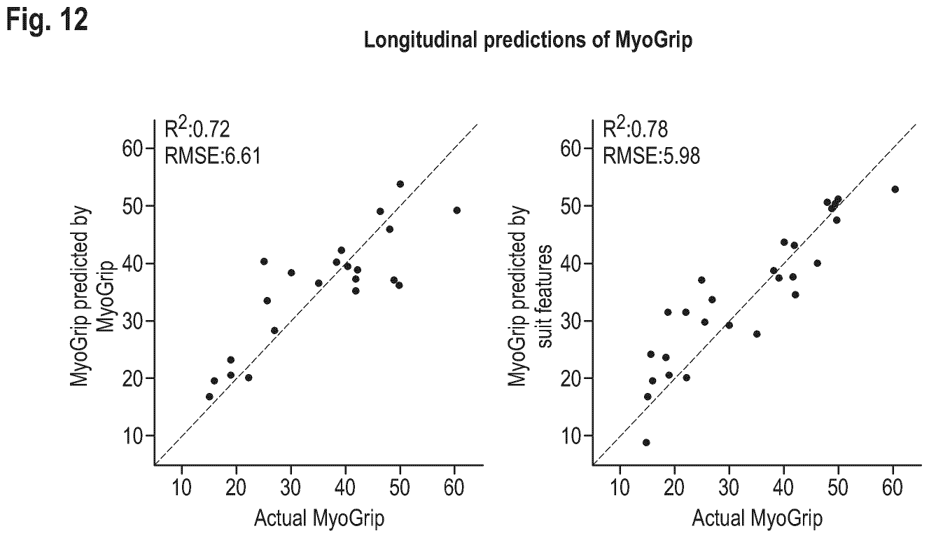

FIG. 12 shows the Longitudinal predictions of Myogrip, using respectively Myogrip and the suit features.

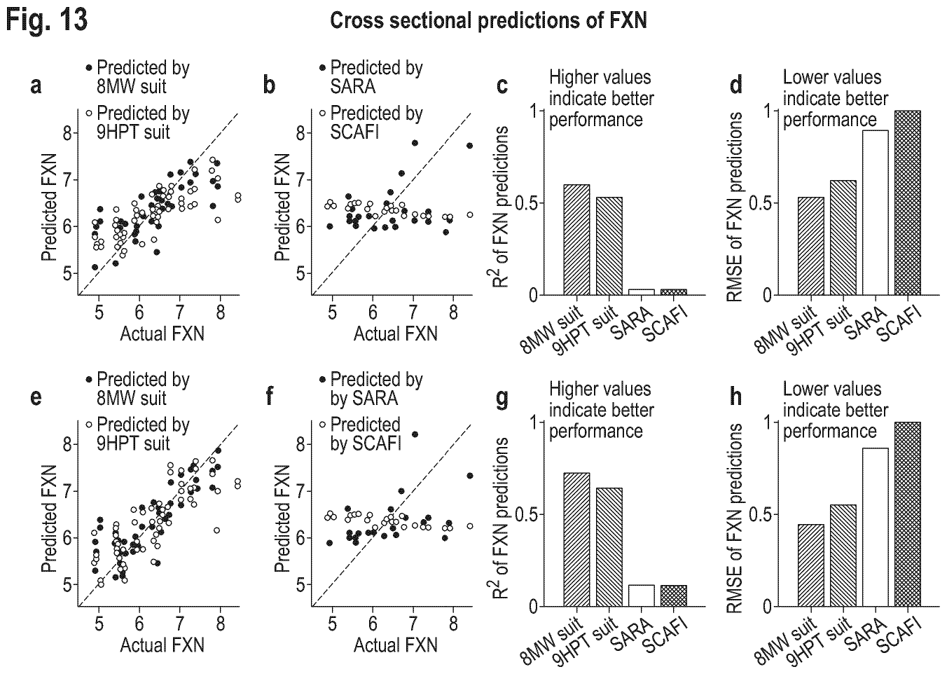

FIG. 13 shows cross sectional predictions of FXN.

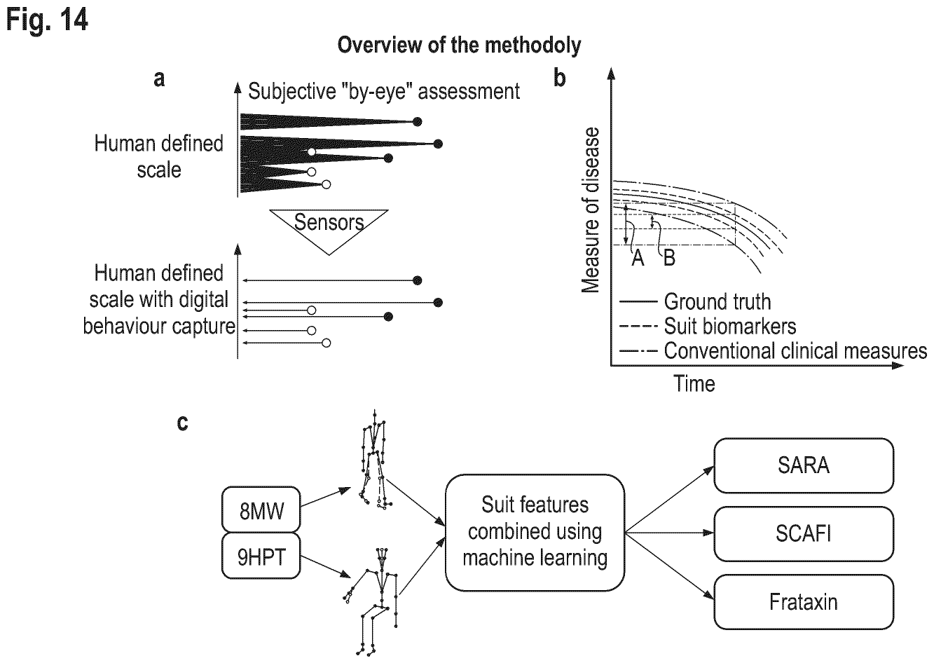

FIG. 14 shows an overview of the methodology followed by the inventors.

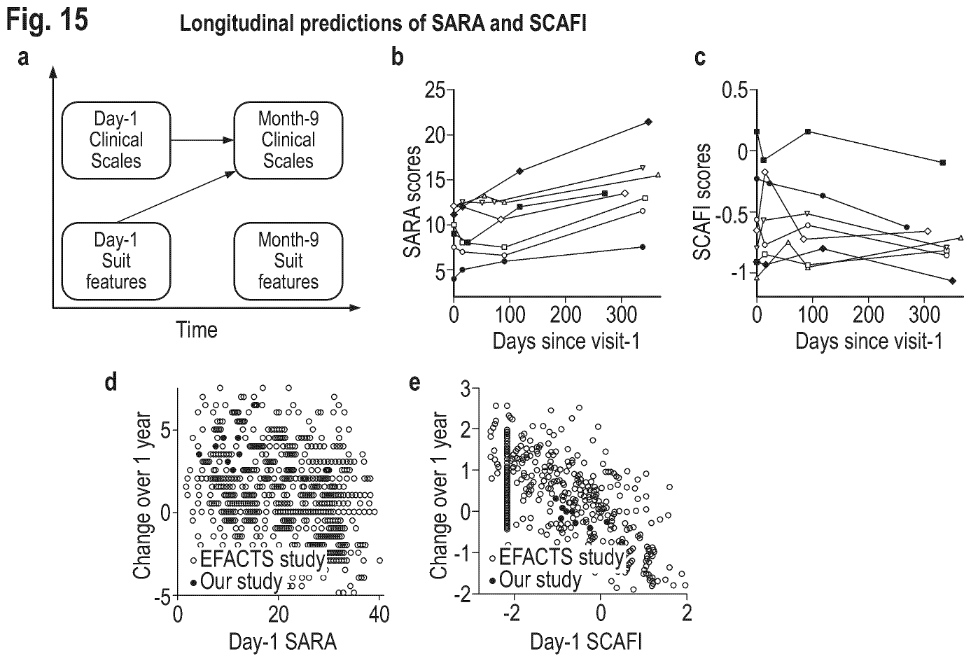

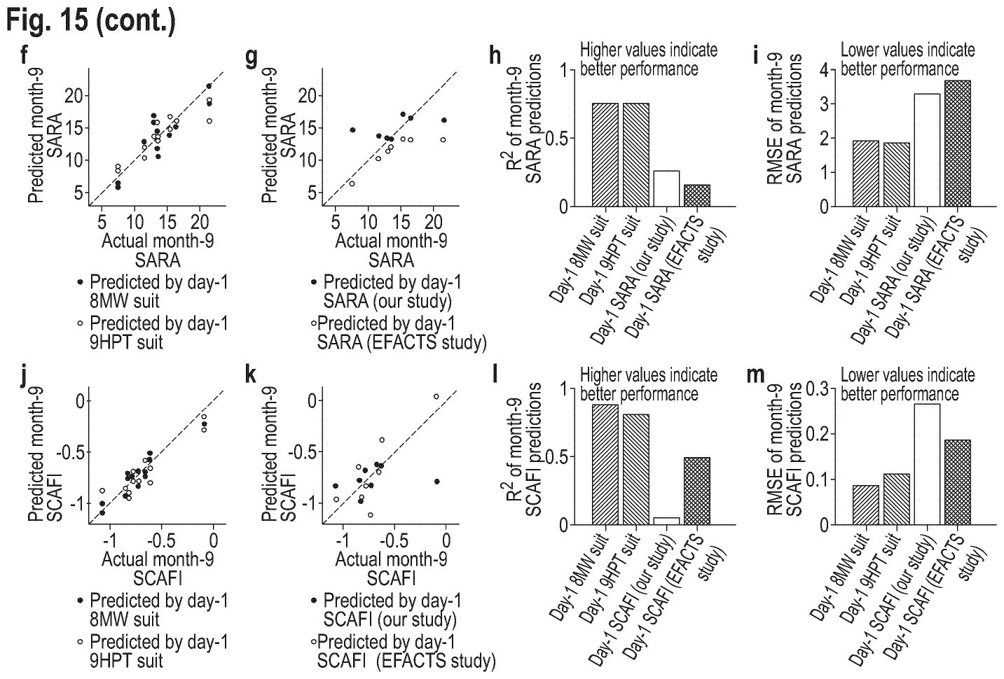

FIG. 15 shows Longitudinal predictions of SARA and SCAFI.

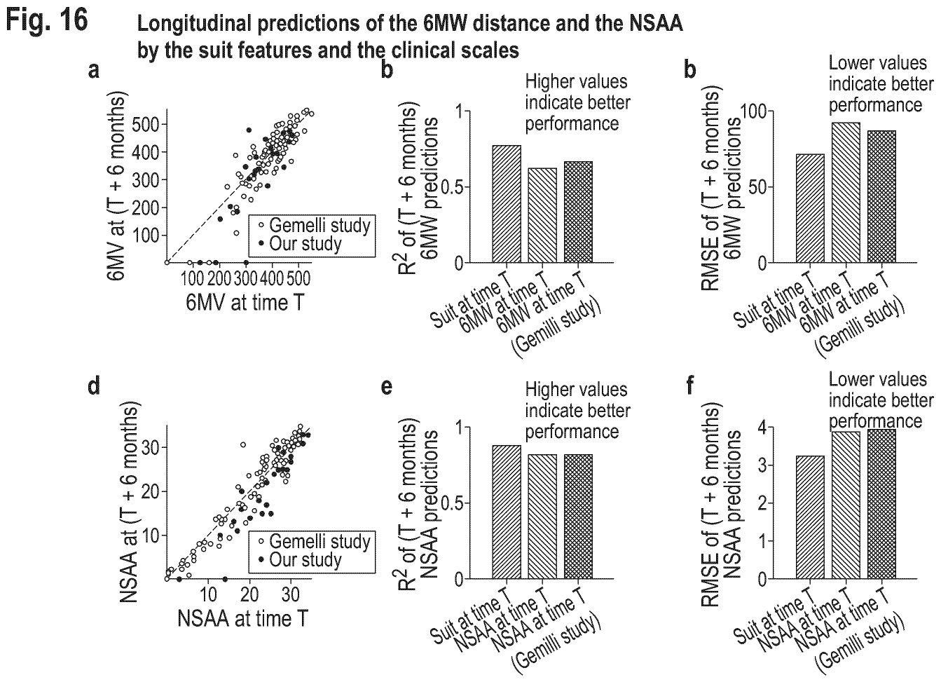

FIG. 16 shows longitudinal predictions of the 6MW distance and the NSAA by the suit features and the clinical scales.

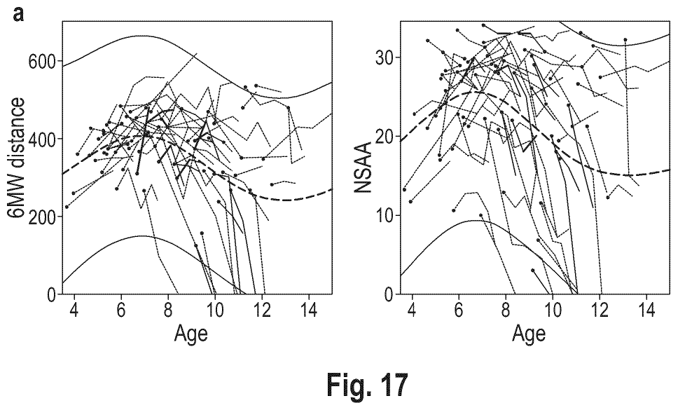

FIG. 17 shows how conventional clinical scales change with age.

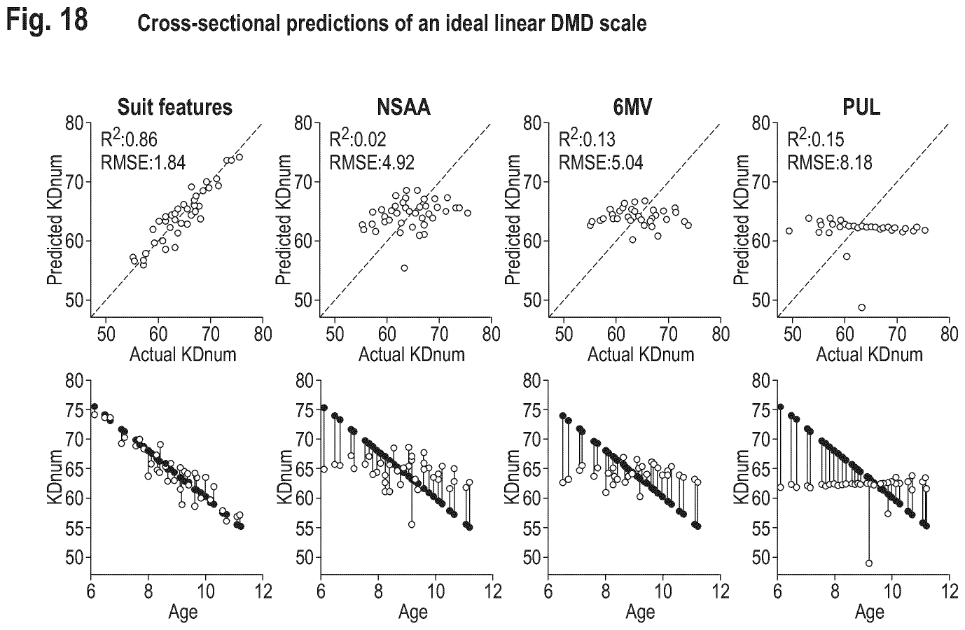

FIG. 18 shows cross-sectional predictions of an ideal linear DMD scale.

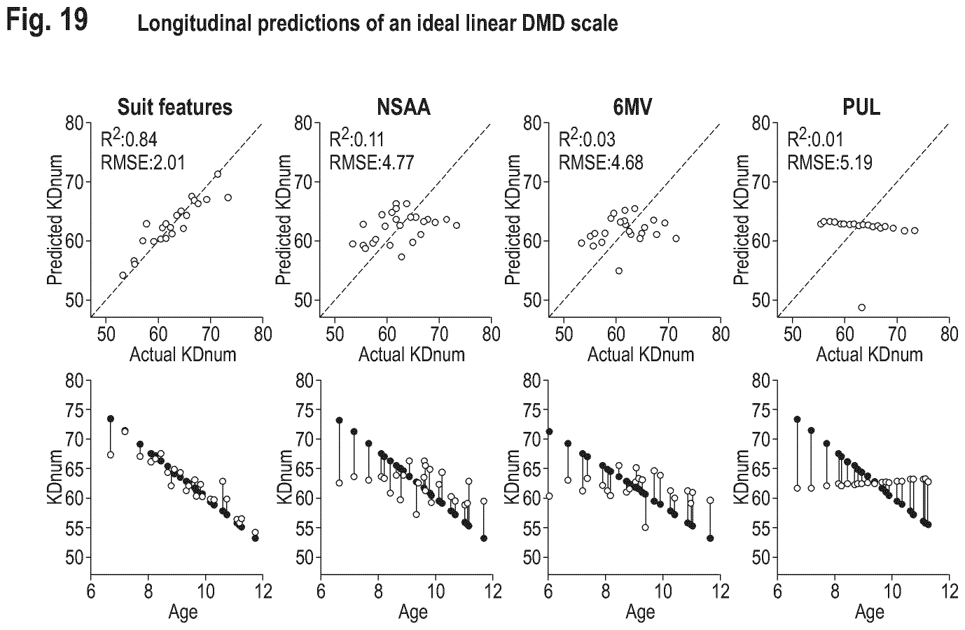

FIG. 19 shows longitudinal predictions of an ideal linear DMD scale.

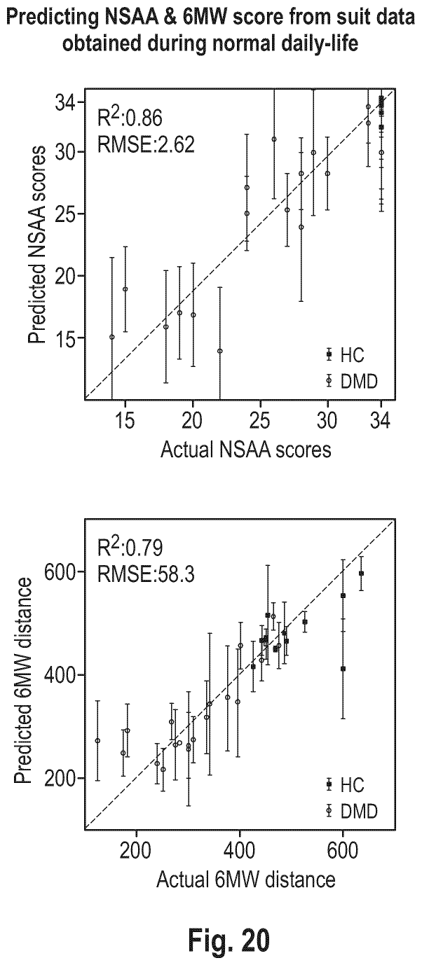

FIG. 20 shows the cross-sectional prediction of the NSAA and 6MW scale from suit data obtained during daily life activity—DMD and healthy control children in a semi-public space with cafeteria and play areas.

In the following description, the present invention will be described with reference to biomarkers for a disease, including one of a neuromuscular, neurological, neurodegenerative, psychiatric and musculoskeletal disease. However, all methods and systems described herein can be likewise applied for identifying or making use of biomarkers for detecting, more generally, the state of a person, including well-being, sense of fitness, or other diseases like brain or bone cancer, diabetes and other endocrinological disorders, epilepsy, dystonia, ataxia, cardiovascular disease or cardiopulmonary disease and a wide range of degenerative system diseases, genetic or acquired such as arthritis. The state of a person may also include a level of fitness out of several different levels of fitness. The state of a person may also include the expression (e.g., the level of expression), of a human gene, e.g., in Friedreichs Ataxia of the Frataxin gene. The state of a person may also include mental health. The state of a person may be pathological (a disease or disorder) or non-pathological.

Herein, the term “person” generally refers to a human subject, but the term may also be interchanged with nonhuman subjects, e.g., a nonhuman primate. Herein, a “person” may be subject or patient having a particular disease or disorder, or a subject characterised by a pathological or non-pathological state.

FIG. 1 illustrates a method according to the invention for identifying a biomarker of interest for a disease, wherein the disease is one of a neuromuscular, neurological, neurodegenerative, psychiatric and musculoskeletal disease. In particular, the present embodiment is adapted to identify a biomarker, e.g., for muscular dystrophy. More specifically, the present embodiment is adapted to identify a biomarker for Duchenne Muscular Dystrophy.

The method of FIG. 1 comprises the following steps:

Step S1: Determining a candidate biomarker.

In step S1, the candidate biomarker can be for example a joint workspace. More specifically, the candidate biomarker can be the knee workspace, as shown in FIG. 3 with reference number 131. Alternatively, the candidate biomarker can be the ankle workspace, as shown with reference number 130 in FIG. 3. Also, the hips workspace might be considered as a candidate biomarker, see reference number 132 in FIG. 3. Alternatively, the candidate biomarker can be the hip orbit during walking or during an instructed movement, as shown in FIG. 4A, reference 140. Alternatively, the candidate biomarker can be the head orbit, as shown in FIG. 4B, reference 141. Different biomarkers can be tested subsequently in order to identify a most suitable biomarker for a predetermined disease or state of a person. Other examples of possible biomarkers are the average peak velocity, the joint angle distribution and an extremity velocity profile. The biomarker can be under a certain movement condition such as “during walking” (e.g., workspace of the knee during walking). However, the invention is not limited to this, as the biomarker might be related to any instructed behaviour (or movement) different from walking. The biomarker might also be related to unconstrained normal daily life (see FIG. 20).

Step S2: Obtaining from at least one motion sensor first motion data from at least one person having the disease, e.g. the neuromuscular disease.

Step S3: Obtaining from the at least one motion sensor second motion data from at least one person not having the disease.

In steps S2 and S3, motion data concerning at least a target person (e.g. a person affected by a disease) and a reference person (e.g. a person which is not affected by the disease) is collected using motion sensors. E.g., the first and second motion data may comprise data from the movement of at least one of an ankle, a wrist, a knee, an elbow, a shoulder, an upper arm, a lower arm, a head, an upper leg, a lower leg, a hip/pelvis, the spine and the chest. Motion sensors applied to the body of a person can be used, e.g. 6 degrees of freedom sensors; however, also other types of motion sensors can be used. The motion sensors can include any known motion sensors, and also e.g. a video camera detecting motion of a person. Further, the motion data obtained by the motion sensor can be collected by optical motion tracking, video-based motion tracking, any form of capture of motion and movement that relates back its data to skeletal movement. According to a preferred embodiment, motion data collected from a plurality of persons is collected, specifically a plurality of persons with disease and a plurality of persons without the disease.

Step S4: Calculating at least a first value related to the candidate biomarker based on the first motion data and at least a second value related to the candidate biomarker based on the second motion data.

The value related to the candidate biomarker can be e.g. the value of the biomarker, such as the workspace volume of the knee during walking. For example, in FIG. 6A the workspace volume distribution for person with the disease (FRDA and DMD) and for healthy control (HC) persons is illustrated.

Step S5: Determining based on the first and the second values of the candidate biomarker whether the candidate biomarker distinguishes between the at least one person having the disease and the at least one person not having the disease.

Based on the difference between the value related to the biomarker deriving from persons with disease and the value related to the biomarker deriving healthy persons (see e.g. FIG. 6A), the method determines whether the candidate biomarker distinguishes between the at least one person having the disease and the at least one person not having the disease.

Step S6: Obtaining first clinical score data according to a conventional clinical score protocol from the at least one person having the disease.

Step S7: Obtaining second clinical score data according to the conventional clinical score protocol from at the least one person not having the disease.

The conventional clinical score protocol may be, e.g., at least one of 6 Minute Walking Test (6MWT), North Star Ambulatory Assessment Scale (NSAA), Scale for the Assessment and Rating of Ataxia (SARA) and SCA Functional Index, or other clinical score protocols mentioned herein or known in the art. In the examples of FIGS. 6B and 6C, 6 Minute Walking Test and North Star Ambulatory Assessment total score are considered. The first and second clinical score data could be obtained using an AI algorithm.

Step S8: Determining a level of correlation between a set of the calculated first and second values of the candidate biomarker and a set of the obtained first and second clinical score data.

As shown e.g. in FIGS. 6B and 6C, based on the set of the calculated first and second values of the candidate biomarker (preferably from a plurality of persons with disease and without disease) and a set of the obtained first and second clinical score data, the method determines a correlation or a level of correlation between the values of the candidate biomarker and the obtained first and second clinical score data according to known protocols.

Step S9: Identifying, based on the result of the first determination step and on the level of correlation, whether the candidate biomarker is a biomarker of interest.

Using the information determined at steps S5 and S8, the method can identify whether the candidate biomarker is a suitable and good biomarker for a specific disease (or more generally for a state of a person, including well-being, state of fitness and so on).

It should be noted that the method steps S1-S9 could be performed also in a different order compared to the order in which they are described above.

In one embodiment, the step S2 of obtaining motion data from at least one person having the disease includes obtaining first motion data from a group of persons having the disease. The people in the group of persons having the disease are heterogeneous with respect to one or more confounding factors, also referred to herein as “confounders”. Further, the step S3 of obtaining second motion data from at least one person not having the disease includes obtaining second motion data from a group of persons not having the disease or the other state. The people in the group of persons not having the disease or the other state are heterogeneous with respect to one or more confounders.

The expression “heterogeneous with respect to one or more confounders” may refer to “uniformly (or randomly) distributed across different levels or values of the confounders”. The confounder, as also discussed later, can be seen as any factor that is not related to a disease to be predicted (the disease of interest). The confounder may however affect the results of conventional method for predicting the state of a disease. For example, the confounder can be any of: age, weight, height, sex, diet, genotype, a second disease other than the disease of interest, or an addiction. However, the invention is not limited to these confounders and may consider any other confounder.

Thanks to the above feature of the method, the present invention allows the identification and use of one or more optimal biomarkers that are robust in use to predict a disease, whereby the prediction is not negatively affected by factors that are not related to the disease of interest. Accordingly, the method and the system making use of the suit features or biomarkers according to the present invention are capable of filtering out influences of one or more circumstances that are not related to a disease, such as (but not only) age, weight, height, sex, diet, genotype, a second disease other than the disease, or an addiction. Therefore, a better prediction of the state of the disease can be achieved under a wide range of circumstances.

The methods and systems described herein for identifying a biomarker of interest can, more generally, be applied to identify a biomarker relevant for detecting, evaluating or predicting a state of a person, which may also include a level of fitness or the like. As described above, the methods and systems making use of the suit features or biomarkers according to the present invention are capable of filtering out influences of one or more circumstances that are not related to the state of interest. Therefore, a better prediction of the state can be achieved under a wide range of circumstances

FIG. 3 shows a system for monitoring the state of a disease using a biomarker. Preferably, the disease is one of a neuromuscular, neurological, neurodegenerative, cardiac, psychiatric and musculoskeletal disease. However, as discussed above, other diseases can also be monitored, or more generally a state of a person, including, e.g., mental health or a nonpathological state not relating to a disease.

The system 100 comprises a motion data obtaining unit 110 configured to obtain from at least one motion sensor 120 motion data from a person having the disease. Preferably, a plurality of motion sensors 120 can be used to provide motion data to the motion data obtaining unit 110. The motion data obtaining unit 110 can be implemented, e.g., with a wireless receiver or/and any cabled input port. The motion sensors can include any kind of motion sensor, including also video camera, wearable sensors, image tracking system or the like as discussed above with reference to the embodiment of FIG. 1.

The system further comprises a generating unit 111 configured to generate a value of the biomarker of the disease based on the obtained motion data. For example, the biomarker can be the knee workspace. In this case, the generating unit calculates the volume defined by the positions of the knee during the detected motion of the target person.

The system 100 further comprises an assessing unit 112 configured to assess the value of the biomarker of the disease and, based on the assessment, to output information related to the state of the disease. E.g., the assessing unit 112 may use a function optimized in view of past detections of a disease state based on the biomarker, so as to assess the state of the disease on the basis of biomarker. It has been found that in particular the joint workspace (during walking or during an instructed movement) and the hips orbit are particularly useful to detect the progress or the regression of disease (e.g., FRDA and DMD). Also the average peak speed of extremities of the person (wrists and ankles) has been found to be particularly significant for the purpose of assessing disease, in particular neuromuscular diseases, such as FRDA and DMD.

The present invention has the advantage of improving subjective assessment of clinical endpoints by data-driven objective measurements and analytics. More specifically, thanks to the method of identifying a biomarker of interest for a disease, it is possible to select a biomarker particularly suitable to detect and monitor the development of a disease, such as DMD, but also other diseases as described herein.

Furthermore, thanks to the newly identified biomarkers, it is possible to improve the monitoring of the progress/regression/state of a disease. This makes it possible to test more easily and on a larger scale new therapies for curing a disease, such as FRDA and DMD. Furthermore, by selecting biomarkers out of a plurality of possible biomarkers, it is possible to select biomarkers which can be detected with low burden for the person having a disease. E.g., it is possible to monitor the progress of a disease with a non-intrusive motion sensor suitable to obtain the value of an identified biomarker.

The method and the systems of the present invention may make use of techniques used in two projects: The first project is “The Longitudinal pharmacodynamics study for the epigenetic effects of Nicotinamide in Friedreich's Ataxia” run by Imperial College London. This Friedreich's Ataxia project uses devices such as surface sensors to monitor movement in children with Friedreich's Ataxia (FRDA) and normal age-matched healthy controls (HC) both in a hospital setting (standard tasks such as 8 meter test etc) and in the home/hospital room environment (24/7). The Nicotinamide study pursued a patient-centric approach to obtain a complete data-driven picture of full-body behavioural capacity in real-life over a 12 month period. The approach of the present invention involves two sides: “At home” & “In clinic”. “In clinic” we focus on full-body motion analysis using high-resolution wearable sensors that can track full-skeleton movements of patients during clinical assessments, therefore reducing subjective assessment of clinical endpoints by data-driven objective analytics. “At home” we focus on a number of wearables to assess the disease progression using just 4 or less sensors placed on the extremities of the children.

This was followed by the second project, KineDMD, a project jointly run by Imperial College London and Great Ormond Street Hospital BRC. The KineDMD study followed broadly the approach established in the Nicotinamide study to monitor movement in children with Duchenne Muscular Dystrophy (DMD) and normal age-matched healthy controls (HC) both in a hospital setting (standard tasks such as 6 min walk test etc) and in the home environment (24/7). The KineDMD study also pursues a patient-centric approach to obtain a complete data-driven picture of full-body behavioural capacity in real-life over a 12 month period. The general approach of the present invention involves two sides: “At home” & “In clinic”. “In clinic” we focus on full-body motion analysis using high-resolution wearable sensors that can track full-skeleton movements of patients during clinical assessments, therefore reducing subjective assessment of clinical endpoints by data-driven objective analytics. “At home” we focus on a number of wearables to assess the disease progression using just 4 or less sensors placed on the extremities of the children.

In the systems and methods according to the present invention, a commercial full-body motion capture suit containing 17 sensors can be used (see FIG. 9). The anatomical landmarks where each sensor is located cover all of the significant areas on the body including each upper and lower limb, each hand, each foot, each shoulder, the head, the pelvis and the middle of the spine. From this the commercial software reconstructs the configuration or pose of the human skeleton. It is this skeletal data that is analysed with the algorithms of the present invention.

The features of the data for both the clinical assessments (“suit”) and natural behaviour (“wearables”) can predict disease progression as well as regress gold-standard clinical scores from sub-measurements of clinical assessments.

The invention comprises multiple components applicable to potentially all clinical trials in Duchenne muscular dystrophy but also to all neuromuscular and neurodegenerative diseases.

DMD boys and age matched healthy controls (HC) are assessed in clinic during 6-monthly visits in conjunction with physio assessments whilst wearing 17 sensors, velcro-attached to clothing, capturing full body kinematics of 22 joints.

FIG. 4C shows the workspace volume for the experimental group having FRDA and the HC group. Herein, workspace volume describes a volume that is encompassed during the measurement period by the measured person inferred from the measured data. The volume of the workspace is highly correlated with disease state (FRDA) and correlates well with disease gold-standard biomarkers, e.g. as predicted using AI technology.

From FIG. 4C, one sees that there is a significant difference between the workspace volume of the two groups “Patients” and “Controls”, indeed, the workspace volume of the control group is significantly smaller.

FIG. 5A shows the average peak velocity for different sensor locations and different groups of measured persons. An average peak velocity describes an average over all measured peak velocities, i.e. first peaks are extracted from data and then these peaks are average. The different sensor locations shown in FIG. 5A are the dominant (“Dom”) and the non-dominant (“N-Dom”) ankle, the dominant and the non-dominant wrist as well as the neck. Herein, dominant and non-dominant refer to handedness of the measured person, i.e. the dominant wrist or ankle refers to the wrist or ankle that is used preferably by the measured person compared to the non-dominant wrist or ankle. The different groups of measured persons refer to an experimental group of persons having the Duchenne Muscular Dystrophy (DMD) disease and to a healthy control (HC) group.

However, the present invention is not limited to these locations; indeed, other locations for sensor placement are conceivable as well. Furthermore, also different measurement systems that do not need sensors placement on the body of the measured person, such as video cameras, optical systems, and the like are conceivable. Furthermore, such measurements are also not limited to the DMD disease but instead can be generalized to other diseases and states of a measured person.

From the data presented in FIG. 5A one understands that for a plurality of sensor locations data can be obtained that clearly differentiates between the experimental group and the control group and further that different sensor locations provide differently strong indications of this differentiation.

FIG. 5B shows a graph of the average peak velocity for various sensor locations between patient and controls in FRDA.

FIG. 6A shows the workspace volume during walking for the experimental group having DMD and the HC group. Herein, workspace volume describes a volume that is encompassed during the measurement period by the measured person inferred from the measured data. The volume of the workspace is highly correlated with disease state (DMD) and correlates well with disease gold-standard biomarkers, e.g. as predicted using AI technology.

From FIG. 6A, one sees that there is a significant difference between the workspace volume of the two groups “DMD” and “HC”, indeed, the workspace volume of the HC group is significantly bigger.

FIG. 6B shows measurement points of the 6MW distance and the workspace volume for several measurements among both groups, “DMD” and “HC”, as well as a linear regression of the measured data.

FIG. 6C shows measurement points of the NSAA score and the workspace volume for several measurements among both groups, “DMD” and “HC”, as well as a linear regression of the measured data.

In combinations, FIGS. 6A-C show that the volume of the workspace is highly correlated with the disease state (DMD).

FIG. 6D shows the workspace volume during walking for an experimental FRDA group and a control group.

FIG. 7A show the area of the orbit of the hips during walking for the experimental group and the HC group. Herein, area of the orbit of the hips describes the area encompassed during the measurement period by the measured person inferred from the measured data. The orbit of the hips of the is highly correlated with disease state (DMD vs HC) and correlates well with gold-standard disease biomarkers, e.g. as predicted using AI technology.

Also from FIG. 7A one sees that there is significant difference between the area of the DMD group and the HC group as the area of the HC group is significantly smaller.

FIG. 7B shows measurement points of the 6MW distance and the area for several measurements among a DMD group and a HC group, as well as a linear regression of the measured data.

FIG. 7C shows measurement points of the NSAA score and the area for several measurements among both groups, “DMD” and “HC”, as well as a linear regression of the measured data.

FIG. 8 shows various graphs showing measurement points of a clinical score and a measure velocity for the two different groups DMD and HC. The upper row shows as a clinical score a 6MW distance, the lower row shows as a clinical score an NSAA score. The first column (from the left) shows the velocity of the dominant ankle, the second column shows the velocity of the non-dominant ankle, the third column shows the velocity of the dominant wrist, the fourth column shows the velocity of the non-dominant wrist. All graphs include a linear regression of the measured data. Moreover, the extremities (wrist, ankle) velocities are both highly correlated with disease state (DMD vs HC) as well as with disease biomarkers, e.g. as predicted using AI technology.

FIG. 9 shows a side view (left) and a back view (right) of a person, indicating various of the sensor placement positions proposed in the present invention. These include, but art not limited to 1. Pelvis (P), 2. Location on the Thorax or Spine (TX), 3. Head (H), 4. Right Should (RS), 5. Right Upper Arm (RUA), 6. Right Forearm (RFA), 7. Right Hand (RH), 8. Left Shoulder (LS), 9. Left Upper Arm (LUA), 10. Left Forearm (LFA), 11. Left Hand (LH), 12. Right Upper Leg (RUL), 13. Right Lower Leg (RLL), 14. Right Foot (RF), 15. Left Upper Leg (LUL), 16. Left Lower Leg (LLL), 17. Left Foot (LF).

The table here below shows various configurations of sensors used for measurement. In detail, seventeen different configurations with an increasing number of sensors are listed including the specific selection of sensors using the abbreviations also used in FIG. 9.

| Number | |
|---|---|
| Sensor. | Name of Sensor. |
| 1. | RH |
| 2. | TX, RH |
| 3. | TX, LFA, LLL |
| 4. | TX, LH, RLL, LF |
| 5. | P, TX, RH, RLL, RF |
| 6. | P, TX, H, LUA, RF, LLL |
| 7. | P, TX, H, RUA, LUA, LUL, LLL |
| 8. | P, TX, RS, LH, RUL, RLL, LLL, LF |
| 9. | TX, H, RH, LS, LUA, RLL, RF, LUL, LLL |
| 10. | P, TX, H, RUA, RFA, LUA, RUL, RLL, LLL, LF |
| 11. | P, TX, H, RUA, RFA, LUA, LFA, RUL, RLL, RF, LF |
| 12. | P, TX, H, RUA, RFA, LUA, LFA, RUL, RLL, RF, LUL, LLL |
| 13. | P, TX, RS, RUA, RFA, LS, LUA, LFA, RUL, RF, LUL, LLL, LF |
| 14. | P, TX, H, RUA, RFA, LS, LUA, LFA, RUL, RLL, RF, LLL, LF |
| 15. | P, TX, H, RUA, RFA, RH, LS, LUA, LH, RUL, RLL, RF, LUL, LLL, LF |
| 16. | P, TX, H, RS, RUA, RFA, RH, LS, LUA, LFA, RUL, RLL, RF, LUL, LLL, LF |
| 17. | P, TX, H, RS, RUA, RFA, RH, LS, LUA, LFA, LH, RUL, RLL, RF, LUL, LLL, LF |

Further features that can be used for a full body analysis comprise the following:
1. Workspace Probability Density Volume for all the joints—Volume occupied by the joints calculated using the 3D location of the joints
2. Channel delay cross-correlation—The array of eigenvalues of the channel delay cross-correlation matrix of joint angular velocities
3. Endpoint velocity (left hand, right hand, left foot, right foot and neck)—Calculated using the 3D position of the joints in space
4. Area of the right-hip movement in the three planes (similar to the head movement area for FRDA)—Area generated by the hip in the 3 planes
5. Average joint angular velocities—Average of the joint angular velocities
6. Autocorrelation Half-Width of the joints—The width of the autocorrelation curve (of the joint angular velocities) at the point when it reaches a value of 0.5
7. Root mean square power spectrum of the joints—Root mean square of the power distribution of the joint angular velocities
8. Correlation-coefficient of the joint velocities—Pearson's correlation coefficient of the angular velocities of the joint
9. Variability of the joint angle velocities—Scale parameter of the logistic distribution of the joint angular velocities

The above features can be used as biomarkers or candidate biomarkers in the systems and methods according to the present invention.

In the present description, the condition “during walking” with regard to a biomarker is not limiting the scope of the invention. Any biomarker described herein may be related to any instructed behaviour (or movement) even other than walking.

The motion sensors described herein can include any known motion sensors, and also e.g. a video camera detecting motion of a person. Further, the data obtained by the motion sensor can be collected by optical motion tracking, video-based motion tracking, any form of capture of motion and movement that relates back its data to skeletal movement.

According to a further embodiment, the invention proposes a method of diagnosing a disease, based on a biomarker, wherein the biomarker is a knee workspace, wherein preferably the disease is one of a neuromuscular, neurological, neurodegenerative, psychiatric, musculoskeletal disease or Friedreich's ataxia.

According to a further embodiment, the invention proposes a method of diagnosing disease based on a biomarker, wherein the biomarker is a joint workspace, wherein preferably the disease is one of a neuromuscular, neurological, neurodegenerative, psychiatric, musculoskeletal disease or Friedreich's ataxia.

According to a further embodiment, the invention proposes a method of diagnosing a disease based on a biomarker, wherein biomarker is an average peak velocity, wherein preferably the disease is one of a neuromuscular, neurological, neurodegenerative, psychiatric, musculoskeletal disease or Friedreich's ataxia.

According to a further embodiment, the invention proposes a method of diagnosing a disease based on a biomarker, wherein biomarker is a joint angle distribution, wherein preferably the disease is one of a neuromuscular, neurological, neurodegenerative, psychiatric, musculoskeletal disease or Friedreich's ataxia.

According to a further embodiment, the invention proposes a method of diagnosing a disease based on a biomarker, wherein the biomarker is an extremity velocity profile, wherein preferably the disease is one of a neuromuscular, neurological, neurodegenerative, psychiatric, musculoskeletal disease or Friedreich's ataxia.

According to a further embodiment, the invention proposes a method of diagnosing a disease based on a biomarker, wherein the biomarker is a hip orbit, wherein preferably the disease is one of a neuromuscular, neurological, neurodegenerative, psychiatric, musculoskeletal disease or Friedreich's ataxia.

According to a further embodiment, the invention proposes a method of diagnosing a disease based on a biomarker, wherein the biomarker is a hip orbit during walking, wherein preferably the disease is one of a neuromuscular, neurological, neurodegenerative, psychiatric, musculoskeletal disease or Friedreich's ataxia.

According to a further embodiment, the invention proposes a method of diagnosing a disease based on a biomarker, wherein the biomarker is a head orbit, wherein preferably the disease is one of a neuromuscular, neurological, neurodegenerative, psychiatric, musculoskeletal disease or Friedreich's ataxia.

A biomarker may be a set of data related to positions of one or more points of the body of the person during a predetermined movement of the person (e.g. during walking). This set of data may describe or represent an orbit, a curve, a trajectory, a volume, a workspace, a distribution, a space distribution or a related derived quantity such as a velocity profile. An example of a biomarker can thus be a data structure suitable to represent an object such as an orbit, a curve, a trajectory, a volume, a workspace, a distribution, a space distribution or a related derived quantity such as a velocity profile. The value of a biomarker may be one or more specific quantities identifying a specific object represented by the biomarker. Thus, the value of a biomarker may correspond to one or more specific quantities characterizing a data structure. The value of the biomarker can be generated based on motion data obtained by one or more motion sensors detecting a movement of the person or a movement of one or more points of the body of the person.

A biomarker can be specified under the condition “during walking”, e.g. hip orbit during walking, head orbit during walking or joint workspace during walking. In this case, the biomarker might be specified with respect to a reference point of the person, by filtering out the movement of the reference point. However, the present disclosure is not limited to this, and the biomarker may also be determined without filtering out the displacement of a reference point of the person during walking. Any biomarker described in the present disclosure may be under the condition “during walking” or “during an instructed movement” or “during a predetermined movement”.

As described above, the biomarker can be an orbit. In this case, the biomarker may correspond to the trajectory of a certain point of the body of the person, e.g. a certain point of the hip (in case the biomarker is a hip orbit) or of the head (in case the biomarker is a head orbit).

Furthermore, in case the biomarker is a workspace, the biomarker may be a volume whose boundary is defined by the positions occupied by a certain point of the body of the person (e.g. a point of the knee of the person) or by the positions occupied by a portion of the body of the person. However, the present disclosure is not limited to this and may encompass also any other workspace, including e.g. a set of discrete positions or spaces occupied by a point or by a portion of the body of the person. In the present disclosure, the term “workspace” may be interchangeable with the expression “workspace volume”.

Furthermore, the biomarker may be a distribution, such as a joint angle distribution. Thus, the biomarker is not limited to data localised in space (such as a curve or a volume), but may also depend on the distribution of the data (including e.g. how often each point of a space is “visited” by a certain point of or by a portion of the body of a person). Further, the biomarker may be an extremity velocity profile and may thus include a quantity derived by the positions of a certain point of the body of a person, such as velocity.

A biomarker may be also a combination of other biomarkers, specifically a combination of any of the biomarkers disclosed herein.

FIG. 10 shows an illustrative implementation of a computer system 300 that may be used to implement one or more of the above described devices, for example to implement the above system 100. The computer system 300 may include one or more processors 310 and one or more non-transitory computer-readable storage media (e.g., memory 320 and/or one or more non-volatile storage media 330). The processor 310 may control writing data to and reading data from the memory 320 and/or the nonvolatile storage device 330 in any suitable known manner. Processor 310, for example, may form and/or perform the functions of the generating unit 111 and the assessing unit 112 of system 100.

To perform the functionality above described of the system 1, the processor 310 may execute instructions stored in one or more computer-readable storage media (e.g., the memory 320, storage media, etc.), which may serve as non-transitory computer-readable storage media storing instructions for execution by processor 310. The computer system 300 includes an input/output functionality 340 to receive data and to provide data, and may include a control apparatus to perform I/O functionality. In particular, the computer system 300, when implementing the apparatus 100, includes one or more antennas for receiving motion data from the one or more sensors 120 or to transmit the information related to the state of the disease e.g. to a display (not shown) or any other output periphery. The system 300 may be implemented as server remote with respect to the motion sensors 120. Furthermore, the system 300 may be implemented with a plurality of distributed processing devices, connected via a network, and/or making use of cloud computing.

In an embodiment, it was possible to reconstruct and identify additional biomarkers, e.g. derived from force production measures (e.g., in Duchenne Muscular Dystrophy: MyoGrip score) and upper body focused measures (e.g., in Duchenne Muscular Dystrophy: PUL score). FIG. 11 shows the longitudinal prediction of PUL, using respectively KD PUL, Gemeli PUL and the “suit features” (the “suit feature” being a non-limiting example of a biomarker of the present disclosure). FIG. 12 shows the Longitudinal predictions of Myogrip, using respectively Myogrip and the suit features.

Thus, according to the invention, further examples of a biomarker may be derived from force production measures or from upper body focused measure. These biomarkers have been found to be particularly useful to predict the state of a disease such as Duchenne Muscular Dystrophy. However, a broader application and use of such biomarkers can be expected to predict other disease or states of a person.

In an embodiment, if was possible to reconstruct/predict the expression of human genes or gene expression levels and related molecular biological data by using a biomarker as described herein. Herein below an example of this application is described in Friedreichs Ataxia, including a demonstration of Cross-sectional prediction of FXN mRNA levels.

FRDA is caused by a GAA-repeat expansion in the FXN gene leading to transcriptional repression of FXN and the disease. This is a gene that is only marginally connected to behaviour, by influencing mitochondrial function in variety of tissues including the brain. Thus, being able to detect changes in FXN is highly indicative of the potency of the biomarkers of the present disclosure.

To demonstrate the ability of the present invention to predict expression levels of the FXN gene, the present inventors measured gene expression levels in tested FRDA patients by RNA extraction and Quantitative Real-time polymerase chain reaction (qRT-PCR), as follows:

Human blood samples were obtained from FRDA patients in accordance with UK Human Tissue Authority ethical guidelines. Peripheral blood mononuclear cells (PBMC) were isolated from the blood samples using a Ficoll-Hypaque TM gradient (Sigma) kit by following the manufacturer's protocol. Total RNA was isolated from the pelleted PBMC using Trizol (Invitrogen) and reverse transcribed using the ThermoScript™ Reverse Transcription system (Invitrogen) by following the manufacturer's instructions. Multiplexed qRT-PCR using TaqMan probes targeting FXN and TATA-box binding protein (TBP) were performed in TaqMan Fast Advanced Master Mix (Applied Biosystems). The measured FXN mRNA levels were expressed relative to TBP as the endogenous control mRNA levels.

The inventors predicted the FXN mRNA levels of the subjects using four sets of predictors: 8MW suit features, SHPT suit features, SARA and SCAFI. The results for the leave-one-subject-out cross-validation are presented in the top row of the FIG. 13 (subplots 4a to 4d) and the results for the leave-one-visit-out cross-validation are presented in the bottom row of FIG. 13 (subplots 4e to 4h).

In more detail, FIG. 13 relates to cross sectional predictions of FXN. FXN mRNA levels were predicted using the following four sets of predictors: suit features from the 8MW task, suit features from the SHPT task, SARA and SCAFI scores. The different panels of FIG. 13 show the following:

a. Scatter plot of the measured FXN mRNA levels against the FXN mRNA levels predicted by the suit features from the 8MW and 9HPT tasks of the corresponding visits.

b. Scatter plot of the measured FXN mRNA levels against the FXN mRNA levels predicted by the SARA and SCAFI clinical scales of the corresponding visits.

c & d. R² and RMSE of the predictions by the 4 set of predictors. GP regression models using the features of the suit data of 8MW and 9HPT tasks as predictors perform better vis-a-vis GP regression models using the SARA and SCAFI scales as predictors.

Results shown in subplots a to d are for leave-one-subject-out cross-validation. The corresponding results for the leave-one-visit-out cross-validation are shown in the bottom row in the subplots e to h.

The scatter-plot of the actual vs predicted FXN levels are presented in the first two columns. The first column shows the predictions using the 8MW & 9HPT suit features and the second column shows the predictions using the SARA and SCAFI clinical scales. 8MW and 9HPT achieved a R² of 0.6 and 0.53 (and a RMSE of 0.53 and 0.62) for the leave-one-subject-out cross-validated case. In comparison, both SARA and SCAFI achieved only R² values close to zero (with RMSE values of 0.89 and 1.00). The R² of the FXN mRNA level predictions by 8MW and SHPT suit features increased to 0.71 and 0.63 (with corresponding RMSEs of 0.45 and 0.55) in the case of leave-one-visit-out cross-validation while the R² of the FXN mRNA level predictions by SARA and SCAFI remained low at 0.11 and 0.11 (with corresponding RMSEs of 0.85 and 1.00).

The specific features that were used for predicting the FXN levels are: the variance in the joint velocity of the knee rotation, the power spectrum of the dominant hip flexion and the non-dominant hip abduction, and the correlation between the dominant and the non-dominant hip flexion joint velocities. There are further examples of biomarkers according to the invention. Any of the variance in the joint velocity of the knee rotation, the power spectrum of the dominant hip flexion and the non-dominant hip abduction, and the correlation between the dominant and the non-dominant hip flexion joint velocities (or a combination thereof) are found useful to predict a human gene expression in Friedreichs Ataxia for Frataxin gene. However, based on the full set of behaviour features and biomarkers disclosed herein, the present invention makes it possible to reconstruct/predict other more removed genetic and epigenetic data using these behavioural features and biomarkers.

In an embodiment of the systems and of the methods for outputting information related to the state of a disease described herein, the information related to the state of the disease may be (or may include) information related to the evolution over time of the disease. In other words, the information related to the state of the disease is information related to the progression of a disease or to a change of the disease. Thus, the biomarkers described herein and the related methods and systems according to the invention also allow one to predict and/or evaluate the evolution or progression of a disease. Specifically, the invention allows one to reconstruct/predict disease evolution better than existing biomarkers and conventional methods. As demonstrated by the experimental results provided herein, the methods and systems of the invention can determine changes in the disease faster and more precisely than existing biomarkers and conventional methods.

The inventors applied, for example, Gaussian Process (GP) Regression algorithm (any non-linear regression algorithm would be suitable) to combine the extracted behavioural features (referred to herein as the suit features of the invention, i.e. the behaviour patterns or biomarkers described herein) and determine their mapping against standard clinical scales. The benefit of GP is that it yields in addition to the prediction also an estimated uncertainty of the prediction, this can be used to weight or prioritise the importance of markers relative to each other. The inventors used a nested cross-validation procedure for feature selection and model evaluation (to avoid leakage of the test data during the feature selection process). The inner cross-validation loop was used for the feature selection and the outer cross-validation loop was used to evaluate the performance of the model. The inventors used a leave-one-subject-out (leave the rows corresponding to all visits of a subject) for both the inner feature selection cross-validation loop and the outer model evaluation cross-validation loop. The inventors used a forward feature selection approach to select the most optimal subset of features.

For s number of subjects with each v visits, the data consists of s×v rows and the outer cross-validation splits the data into s folds (ensuring all the visits of a subject are in a single fold and each fold contains only the rows corresponding to the visits of a single subject). Thus, there are s training and test folds. Forward feature selection of features was done for each of the s training folds using the leave-one-subject-out cross-validation error of the inner cross-validation loop as the objective function and s subsets of features were generated. Following the standard nested cross-validation procedure, the most frequent subset among the s subsets was selected as the optimal subset as the frequency of the subset of features is a measure of the robustness of the subset of selected features to changes in the training data. Finally, the overall performance of the GP regression was evaluated for the selected optimal subset of features using the outer cross-validation for the s test sets. This method ensured that an optimum subset of features is selected without any data leakage of the test set into the training set. This nested cross-validation approach ensured that the test data in each fold of the outer cross-validation loop was never used during feature selection in the inner cross-validation loop and therefore provides a reliable estimate of the model performance. The hyperparameters of the Gaussian process were chosen based on the cross-validation error on the outer nested loop. The predicted values from all the test folds of the outer fold was aggregated and the aggregate root mean squared error (RMSE) and the coefficient of determination (R2) was calculated and reported in the results section.

Exemplary Demonstration in Friedreich's Ataxia

Next it is shown, using the example of Friedreich's Ataxia patients, how the invention outperforms disease prediction using only conventional biomarkers on a large-scale cohort (EFACTS). The EFACTS study data comes from a longitudinal study of a large cohort of Friedreich's ataxia patients from eleven European study sites where the patients were seen at baseline, 1 year, and 2 years and the different clinical scales were recorded. The inventors built GP regression models with different kernel options to predict the clinical score at time (t+N) days given the clinical score at time t and N days. The inventors picked the model which gave the best performance (minimum RMSE) on a 5 fold cross-validated error. The inventors trained the best performing model on the entire EFACTS data and used it to make predictions on the clinical scales from the inventors' study and the results were reported as test errors (out of sample errors).

FIG. 14 is an overview of the methodology:

a: Current gold-standard clinical assessments for monitoring the effects of neurodegenerative diseases such as Friedreich's ataxia (FA) are often measured “by-eye” and lack objectivity. Sensors can help achieve accurate and objective monitoring of patients' behaviour.

b: Because of the lack of precision of the conventional clinical measures to measure the disease, it takes months before they reveal any change in the disease measurement. Motion capture suit biomarkers are more accurate and sensitive digital biomarkers that can detect even small changes in patient performance. Applying behaviour analytics can capture the subtle changes more objectively and in a shorter time span than the conventional clinical measures. This allows the duration of clinical trials to be reduced, which in turn reduces the drug development costs. Similarly in non-clinical applications it can be used to reduce the duration of human state assessments.

c: Using a full-body motion capture approach, the inventors analysed two sub assessments of the SCAFI scale, the 8 MeterWalk (8MW) and the 9 Hole Peg Test (SHPT), for which clinicians only use their duration for estimating the progression of the FRDA disease. The inventors applied a machine learning approach and generated a series of markers of patient performance. Using this data-driven approach the inventors reconstruct the full SARA and SCAFI scores.

FIG. 15 shows longitudinal predictions of SARA and SCAFI:

a. Overview of the idea of longitudinal predictions of the clinical scales. The inventors predicted the month-9 clinical scales using the suit features from the 8MW and 9HPT tasks on day-1 and compared the predictions of the month-9 clinical scales using the clinical scales from day-1.

b & c. Evolution in patients' SARA (b) and SCAFI (c) scores across different visits. Each coloured line represents the plot of the progression of the clinical scales of a single FRDA patient against the days since his first visit. The inventors can observe that the study patient population is heterogeneous with different stages and progression speeds of the disease.

d & e. Scatter plot of the day-1 SARA (d) and SCAFI (e) vs the change in the clinical scale over 1 year for data from our study and EFACTS study, a larger study with a cohort of 302 subjects. The change in the SCAFI score of the patients over a year can be seen to be related to the day-1 SCAFI score.

f to m. The inventors predicted the month-9 SARA and SCAFI scores of the FRDA A patients using the day-1 SARA & SCAFI scores (from the inventors' study and EFACTS study) as predictors and compared against the predictions made using the features of the suit data (from 8MW and 9HPT) on day-1 as predictors.

Subplots f, g, j & k show the actual vs predicted scores and the subplots h, i, l & m show the R² and RMSE of the predictions. The features of the day-1 suit data predict month-9 clinical scale with higher accuracy when compared to the day-1 clinical scales themselves. Furthermore, the predictions from the suit (N=8) are more accurate than a model using the clinical scales from a large natural history cohort (N=302) as predictors.

Exemplary Demonstration in Duchenne Muscular Dystrophy

FIG. 16 shows longitudinal predictions of the 6MW distance and the NSAA by the suit features and the clinical scales:

a. & d. Scatter plot of the clinical scales from the visit at time T against the clinical scales from a visit at time (T+6 months) from both our study (N=21) and Gemelli study (N=92).

b. & e. R² of the predicted clinical scales from a visit at T+6 months using the suit features and clinical scales themselves from the visit at time T as predictors. The features of the suit data predict the clinical scale at T+6 months with higher accuracy when compared to the clinical scales themselves. Furthermore, the predictions from the suit are more accurate than a model using the clinical scales from a large natural history cohort (N=92) as predictors.

c. & f RMSE of the same predictions using the suit features and clinical scales.

Thus, the invention makes it possible to define clinical scales that are optimally suited to a given disease or state of interest and still reconstruct them very well with the behaviour features or the biomarkers described herein. An exemplary demonstration of this is to define a linear scale that decreases as disease progresses. An exemplary application of this is described in the following in case of Duchenne Muscular Dystrophy.

Many known clinical scales are, unfortunately, defined in a way that does not capture meaningful progression of disease in a straightforward manner. FIG. 17 shows how conventional clinical scales change with age. In FIG. 17 it is shown how scores e.g. increase with age, as children grow (which increases their muscle in an age-dependent manner), while disease keeps reducing their muscle compared to a healthy development. A superposition of the effects of the disease and of unrelated natural effects of the growth of the children (an example of a confounding factor) results in a bump in conventional clinical scores, such that those scores fail to reflect true disease progression that starts at birth. Methods based on such conventional clinical scores are thus deficient in this respect.

The present invention overcomes such deficiencies in that it allows, for example in the case of DMD, the definition of an ideal linear scale that at birth has the value 100 and at 25 age has the value 0, decreasing continuously by 4 score points per age (reflecting the genetic/cellular mechanisms behind disease). The inventors then demonstrated that this “postulated” scale cannot be well reconstructed from a combination of multiple conventional clinical scales (NSAA, 6MW, PUL), but can be very well reconstructed from the suit behaviour patterns (“suit features” or biomarkers)—both cross-sectionally and longitudinally. FIGS. 18 and 19 show how well the suit/behaviour features (or biomarkers) can reconstruct the ideal clinical scale. Some form of non-linear regression is needed to link suit feature values to the ideal score (here GP regression).

Specifically, FIG. 17 illustrates the high variability and bumpiness of existing clinical scales (a. 6 minute walk test; b. Northstar Assessment Score) from large cohort data (Gemelli data).

FIG. 18 shows cross-sectional predictions of an ideal linear DMD scale. FIG. 19 shows longitudinal predictions of an ideal linear DMD scale.

Thus, the systems and the methods according to the present invention are capable of outputting information related to the state of a disease that is independent of a confounder. The term “independent of a confounder” expresses that the information relating to a state of interest or a state of a given disease does not substantially vary when a value of the confounder varies, so that the information related to the state of interest or the state of the disease is significant under different circumstances. As above discussed, the confounder can be seen as any factor that is not related to a disease to be predicted or evaluated or, more generally, a state to by predicted or evaluated. The confounder may however affect the results of conventional methods for predicting the state of a disease. For example, the confounder can be any of: age, weight, height, sex, diet, genotype, a second disease other than the disease of interest, or an addiction. Thus, the method and the system making use of the suit features or biomarkers according to the present invention are capable of filtering out the influence possibly due to a circumstance that is not related to a disease or state of interest. Therefore, a better prediction of the state of the disease or a state of interest can be achieved.

In the context of any embodiment of the invention, “motion data” obtained from a person is not necessarily obtained by means of sensors worn by the person, but may also be obtained via a camera, a radar or any other means suitable for capturing motion data. A “motion sensor” may thus also include a camera, a radar or any other means suitable for capturing motion data. Further, in some embodiments, motion data may be obtained from the person at different points in time and a value of a biomarker may be generated based on the obtained motion data at different points in time; in this case, a plurality of values of the biomarker are generated at different points in time, wherein the plurality of values of the biomarker can be used e.g. (but not only) to determine (or output) the information related to the evolution of the disease over time, in case the information related to the state of the disease include this kind of information.

1. A system for monitoring the state of a disease or other state of a person using a biomarker, the system comprising:
a motion data obtaining unit configured to obtain from at least one motion sensor motion data from a person having the disease or other state,
a generating unit configured to generate a value of the biomarker of the disease based on the obtained motion data, and
an assessing unit configured to assess the value of the biomarker of the disease or other state and, based on the assessment, to output information related to the state of the disease or other state.

2. A method of monitoring the state of a disease using a biomarker, the method comprising:
obtaining from at least one motion sensor motion data from a person having the disease,
generating a value of the biomarker of the disease based on the obtained motion data, and
assessing the value of the biomarker of the disease and, based on the assessment, outputting information related to the state of the disease.

3. A method of monitoring a biomarker of an individual to be tested for a disease or having a disease, the method comprising:
obtaining from at least one motion sensor motion data from a person having the disease,
generating a value of the biomarker of the disease based on the obtained motion data,
assessing the value of the biomarker of the disease and, based on the assessment, outputting information related to the biomarker.

4. (canceled)

5. The system according to claim 1 wherein the genetic or acquired disease is a neuromuscular, neurological, neurodegenerative, psychiatric, cancer, musculoskeletal, and cardiovascular and respiratory disease.

6. The system according to claim 1, wherein the disease is a muscular dystrophy.

7. The system according to claim 1, wherein the disease is Duchenne Muscular Dystrophy.

8. The system according to claim 1, wherein the disease is Friedreich's ataxia.

9. The method according to claim 2, wherein the biomarker is a knee workspace.

10. The method according to claim 2, wherein the biomarker is a joint workspace.

11. The method according to claim 2, wherein the biomarker is an average peak velocity.

12. The method according to claim 2, wherein the biomarker is a joint angle distribution.

13. The method according to claim 2, wherein the biomarker is an extremity velocity profile.

14. The method according to claim 3, wherein the biomarker is a hip orbit.

15. The method according to claim 3, wherein the biomarker is a hip orbit during walking or an instructed movement.

16. The method according to claim 3, wherein the biomarker is a head orbit.

17. The system according to claim 1, wherein the state of the person is the expression of a human gene.

18. The system according to claim 17, wherein the expression of the human gene is a human gene expression in Friedreichs Ataxia for Frataxin gene.

19-40. (canceled)

41. The system according to claim 1, wherein the information related to the state of the disease is information related to the evolution of the disease over time.

42. The system according to claim 1, wherein the information related to the state of the disease is independent from a confounder.

43. The system according to claim 1, wherein the biomarker is derived from force production measures or from upper body focused measures.

44-49. (canceled)
